(19) United States
(12) Plant Patent Application Publication
(10) Pub. No.: US 2003/0014794 P1
(43) Pub. Date: Jan. 16, 2003

(54) Ilex glabra plant named "WSI"

(76) Inventors: Bill Strain, Athens, AL (US); Charles Wilson, Frankfort, KY (US)

Correspondence Address: Mark A. Taylor, Stoll. Keenon & Park, LLP, Suite 1000, 201 E. Main Street, Lexington, KY, 40507-1380, US

(21) Appl. No.: 09/904,530

(22) Filed: Jul. 12, 2001

(51) Int. Cl. A01H 5/00

(52) U.S. Cl. PLT/247

(57) ABSTRACT

A distinctive cultivar of Inkberry, Ilex glabra, named “WS1”, characterized by a dense, rounded habit; profilse branching, small intemodes (less than 1.0 cm.), compact form with dark green oblanceolate leaves; excellent retention of filiage; small white flowers forming black drupes; prefers moist acidic solis in U.S. plant hardiness zones 4 to 9.

### BACKGROUND OF THE INVENTION

The present invention relates to a new and distinct cultivar of Inkberry plant known as Ilex glabra WS1 and hereinafter referred to by the cultivar name ‘WS1’.

The Inkberry plant is a popular evergreen holly in North America that appears from Nova Scotia south to Florida and west into Texas. The species has widespread adaptability, i.e. in zones 4-9, and is typically found in moist to wet soil areas. Limitations on the use of Inkberry for landscaping include variability in plant size and growth habit and a tendency to lose lower leaves. Among cultivars already described, the cultivars “Compacta”, Nordic”, “Densa” and “Shamrock” are considered to be the most compact. Even these cultivars become tall and usually lose the lower 20-30% of their leaves. There is accordingly a need for a more dense, rounded, and more foliage-retaining Ilex glabra cultivar.

The new cultivar Ilex glabra ‘WS1’ was discovered as a sport mutation of the nonpatented Ilex glabra ‘Nigra’ on a cultivated plant in Athens, Ala. Ilex glabra ‘Nigra’ has a dense, vase shaped habit that has become popular among cultivars commercially available. Ilex glabra ‘Nigra’, like other cultivars, lacks an ability to maintain foliage on the lower portions of the plant. For that reason, improved cultivars are needed that give more dense habit with retention of lower leaves. Ilex glabra ‘WS1’ provides the characteristics that are needed to improve this species. ‘WS1’ has been observed and asexually reproduced from cutting, over a four-year period, with consistent and effective reproduction of the unique rounded habit, short internodes, and lower leaf retention of the original sport.

### SUMMARY OF THE INVENTION

The following characteristics separate ‘WS1’ from other available cultivars.

1. A branching habit that is denser, with a unique rounded shape more similar to Buxus cultivars than that of Ilex glabra.

2. Plants of ‘WS1’ have proven to have a consistent, uniform rounded habit.

3. Plants of ‘WS1’ have an average internode of less than 1.0 cm. As compared to ‘Nigra’ which can have an internode of greater than 2 cm.

4. Plants of ‘WS1’ will maintain a dense rounded habit with less pruning than required by other Ilex glabra cultivars.

5. Winter foliage is darker in color than typical of the species. The market class of the WS1 is Inkberry Holly.

### BRIEF DESCRIPTION OF THE PHOTOGRAPHS

The accompanying color photographs illustrate the overall appearance and foliage color of the new WS1, illustrating the colors as true as is reasonably possible to obtain in color reproductions of this type.

FIG. 1 Unique branching habit of Ilex glabra ‘WS1’. Dense branches remain low to ground with retention of lower leaves.

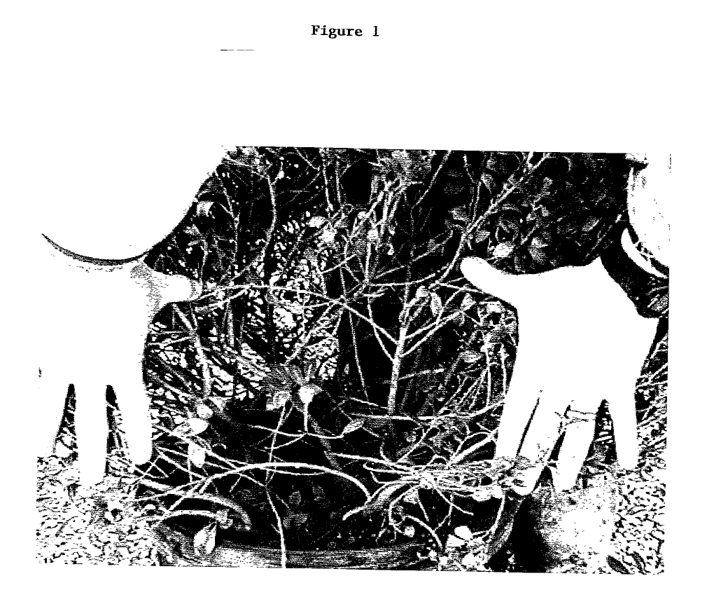

FIG. 2 Branching habit of Ilex glabra ‘Nigra’. Less branching with a more upright habit. Lower portion of the plant has no filiage.

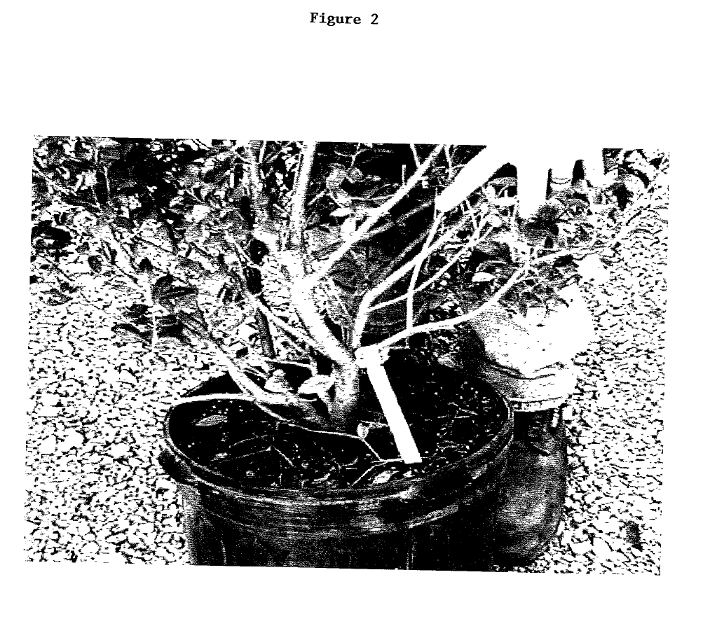

FIG. 3 Branching habit of Ilex glabra ‘Densa’. Less foliage retention than ‘WS1’ or ‘Nigra’ with slender vase shaped trunk.

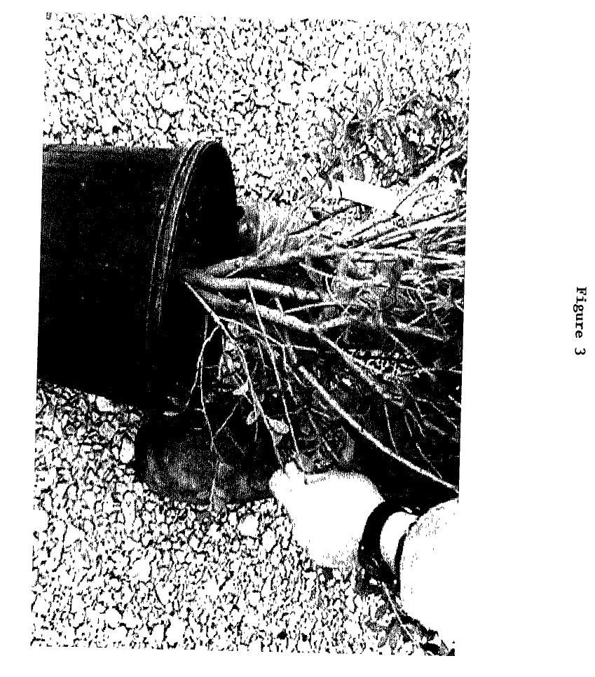

FIG. 4 Stem sections of ‘Densa’(A), ‘Nigra’(b), and ‘WS1’(C). Intemodes on ‘WS1’ are much shorter with twice as many buds. Stem color of ‘WS1’ is less yellow than that of ‘Densa’ or ‘Nigra’.

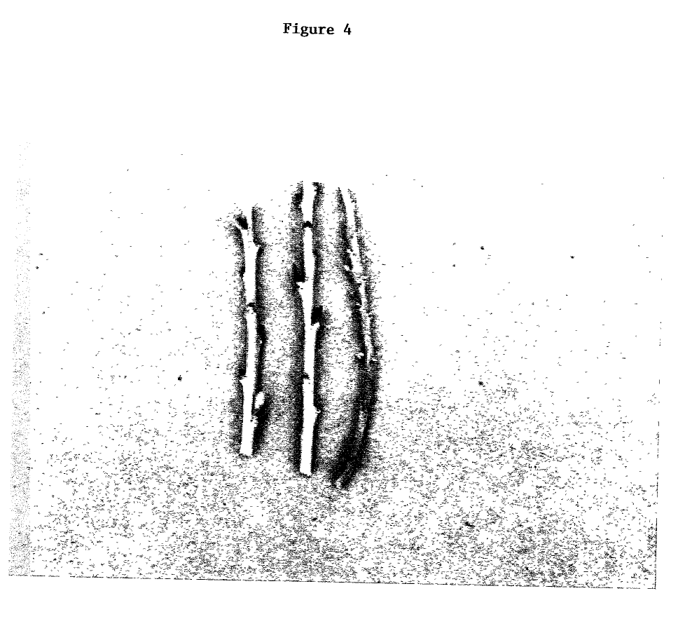

FIG. 5 Comparison of 4-year-old plants of Ilex glabra ‘Densa’(A), ‘Nigra’(b), and ‘WS1’(C). This picture clearly shows the progression of cultivars with ‘WS’ providing the most compact form with retention of lower leaves.

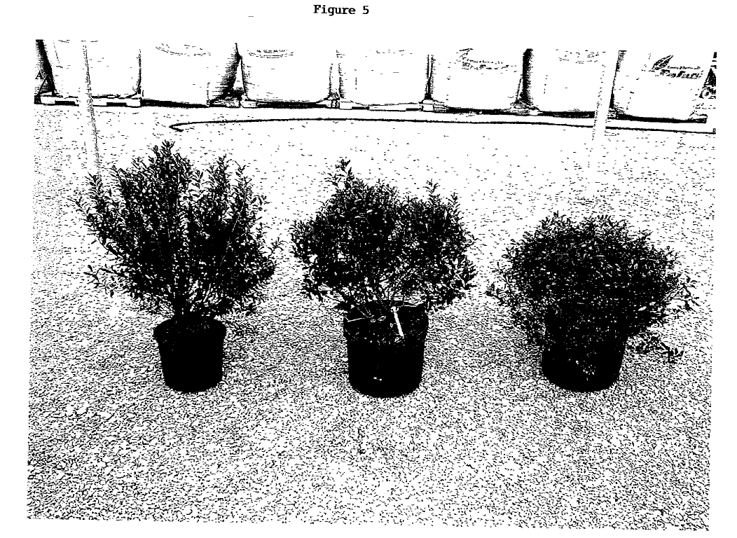

FIG. 6. Typical hardwood cuttings of ‘Densa’(A), ‘Nigra(B), and ’ WS1’(C). Apical cuttings were taken from plants of same age and size. ‘WS1’ is much more compact, producing the lower branching habit.

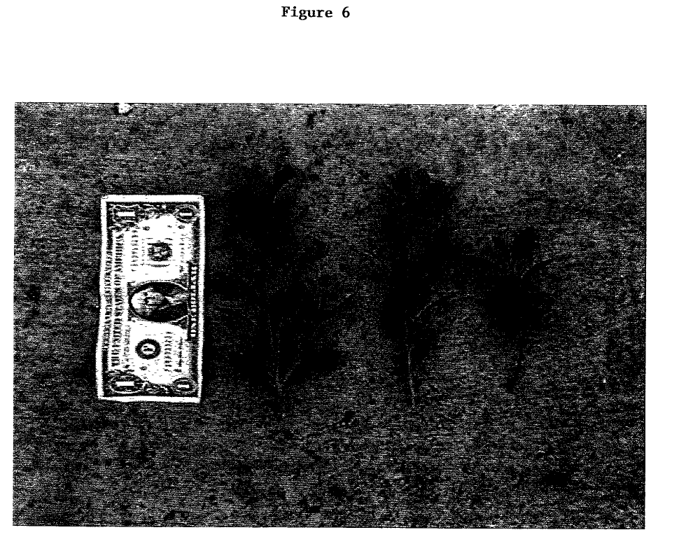

### DETAILED BOTANICAL DESCRIPTION

The following descriptions of ‘WS1’ have been observed under a production environment and found to be consistent in both Athens, Ala., since 1996, and Frankfort, Ky. since 2000. All measurements and descriptions were made from mature plants in accordance with The Royal Horticultural Society Plant Color Chart.

Genus: Ilex

Species: glabra

Cultivar: ‘WS1’

Market Classification: ‘WS1’ Inkberry Holly

Parentage: Sport mutation of Ilex glabra ‘Nigra’.

Propagation: Asexual reproduction by softwood or hardwood cuttings.

Plant form: Dense rounded habit, similar to that of Buxus species, with profuse branching resulting in mature plants equal in height and width.

Plant size: Mature plants will reach a height near 120 cm. With an equal width.

Stem description: Stem texture is smooth when young, maturing to glabrous, ascending, with an internode length of less than 1.0 cm. Young stems are yellow-green (151C), maturing to Gray-Brown(199B).

Foliage description: Leaves are simple, alternate, with entire margins, cuneate with small obtuse teeth near apex, cuneate base, oblanceolate 2-3 cm long, 1.5 cm wide, petiole is 0.6 cm. Long. New foliage is yellow-green (146C), maturing to green (131A).

Flower description: Small white flowers bearing 6 petals emerging in May.

Fruit description: Small berry like drupe, black in color developing in late summer.

Disease resistance: Current plants have displayed no problems from known diseases of Inkberry holly.

It is claimed:

1. A new and distinct Inkberry, Ilex glabra plant named ‘WS1’ as illustrated and described.
